(19) United States
(12) Patent Application Publication
(10) Pub. No.: US 2026/0007580 A1
(43) Pub. Date: Jan. 8, 2026

(54) PULP CAPPING MATERIALS CONTAINING TRICALCIUM SILICATE AND PREPARATION METHODS FOR THE SAME

(71) Applicant: National Taiwan University, Taipei City (TW)

(72) Inventors: Yu-Chih Chiang, Taipei City (TW); Hong-Ping Lin, Taipei City (TW); Hsun-Chang Lin, Taipei City (TW)

(73) Assignee: National Taiwan University, Taipei City (TW)

(21) Appl. No.: 19/236,280

(22) Filed: Jun. 12, 2025

(30) Foreign Application Priority Data
Jul. 5, 2024 (TW) 113125384

(51) Int. Cl. A61K 6/831; A61K 6/78

(52) U.S. Cl. CPC A61K 6/831; A61K 6/78

(57) ABSTRACT

The invention provides a pulp capping material comprising bioactive glass fibers, mesoporous tricalcium silicate, and zirconia dioxide. The initial setting time of this pulp capping material can be reduced to 7 minutes. The material exhibits no significant cytotoxicity and can significantly enhance cell viability. Additionally, it increases the expression of alkaline phosphatase and Dentin Sialophosphoprotein mineralization genes in dental pulp stem cells.

### CROSS-REFERENCE TO RELATED APPLICATION

The present application claims priority to and the benefit of Taiwan Patent Application No. 113125384, filed on Jul. 5, 2024, and entitled “PULP CAPPING MATERIALS AND PREPARATION METHODS AND APPLICATIONS FOR THE SAME”, the contents of which are incorporated herein by reference in their entirety.

### FIELD OF THE INVENTION

The present invention relates to a pulp capping material and a method of preparing the same, and particularly to a calcium silicate-containing pulp capping material for use in the treatment of dental cavities or tooth trauma.

### BACKGROUND OF THE INVENTION

Vital pulp therapy is when the tooth is affected by deep caries or trauma extending to the dental pulp tissue, in which, after the removal of infected and damaged tissue, a minimally invasive procedure using a pulp capping material to block infection and maintain pulp vitality, with the aim of avoiding the shortened lifespan of the affected tooth caused by the greater loss of tooth structure in conventional root canal treatment. Vital pulp therapy includes direct pulp capping, indirect pulp capping, pulpotomy, and the like. Successful vital pulp therapy usually not only maintains pulp vitality, but is also often accompanied by reactionary and reparative dentinogenesis, which is also commonly regarded as evidence of treatment success.

Tziafas has proposed that three factors must be satisfied to achieve a successful prognosis: (1) complete elimination of pulp infection; (2) use of appropriate materials to protect the pulp and induce repair or regeneration; (3) proper sealing and filling to prevent microleakage. An ideal vital pulp therapy material is required to possess sufficient physical and mechanical properties: adequate strength to resist external force, short setting time, and good handling properties; at the same time, it should also offer advantages in terms of biological characteristics: ideal biocompatibility with pulp tissue and the ability to induce mineralized tissue formation.

Conventional technologies still lack an ideal material for vital pulp therapy. In the past, calcium hydroxide or pulp capping materials containing calcium oxide were commonly used in vital pulp therapy. However, both calcium hydroxide and calcium oxide exhibit low mechanical strength and are prone to microleakage. Other issues also persist with commonly used clinical pulp capping materials, such as poor handling properties, prolonged setting time, or the risk of tooth discolouration. A longer setting time necessitates extended waiting periods to ensure complete hardening, which negatively impacts patient experience during treatment. Furthermore, extended chair time or the need for additional appointments increases the risk of secondary infection of the dental pulp.

### BRIEF SUMMARY OF THE INVENTION

The present invention identifies the following disadvantages of using conventional pulp capping material. The main components of conventional pulp capping material powder include nearly half tricalcium silicate (3CaO·SiO₂, C3S), dicalcium silicate (2CaO·SiO₂, C2S), tricalcium aluminate (3CaO·Al₂O₃, C3A), and bismuth oxide with radiopacity, among others. After the conventional pulp capping material powder is mixed with double-distilled water at a weight ratio of 1:0.3, the entire setting process takes approximately 2 hours and 45 minutes. Although conventional pulp capping materials exhibit good biocompatibility, they still suffer from poor handling properties and prolonged setting times. Moreover, the presence of manganese and iron in the composition may lead to discoloration. In addition, a high content of tricalcium aluminate raises concerns regarding tooth discoloration and potential effects on cellular biocompatibility. Therefore, formulations free of tricalcium aluminate are generally viewed more favorably by consumers.

One of the objectives of the present invention is to use tricalcium silicate as a base and combine it with bioactive glass fibers and a radiopacity material to produce a pulp capping material. The pulp capping material of the present invention exhibits mechanical properties that meet expectations of the ability to induce mineralization of the dentin-pulp complex.

The content of the present invention is divided into at least three parts: The first part is the optimization of a tricalcium silicate-based pulp capping material. Synthesized tricalcium silicate was analyzed by X-ray diffraction and observed by scanning and transmission electron microscopy, and its setting time and diametral tensile strength were tested. The second part is in vitro cell compatibility tests, in which the immediate and long-term cell viability of the pulp capping material of the present invention was compared with those of commercially available products. The third part is the investigation of the effect of the pulp capping material of the present invention on the remineralization ability of human dental pulp stem cells. In addition to using alkaline phosphatase quantification and analysis to compare cellular mineralization ability, the inducibility of DENTIN SIALOPHOSPHOPROTEIN (DSPP) and other genes by different material groups was further assessed using quantitative real-time reverse transcription PCR analysis.

The tricalcium silicate synthesized in the present invention is the high purity tricalcium silicate. The precursor of the tricalcium silicate synthesized in the present invention is mesoporous silica with an average pore diameter ranging from 10 nm to 50 nm, preferably from 15 nm to 40 nm, and more preferably from 15 nm to 30 nm. By employing a sol-gel method to produce mesoporous silica with pore sizes within the specified range and using it as a precursor in an impregnation process to synthesize tricalcium silicate, the formation of impurities, particularly calcium oxide, which is unsuitable for use as a pulp capping material, can be effectively avoided.

Dicalcium silicate (C₂S) exhibits a relatively slow hydration rate. In contrast, tricalcium silicate (C₃S) has higher reactivity. In one embodiment, the pulp capping material of the present invention does not contain additional C₂S. Moreover, by controlling the calcium-to-silicon ratio and the pore size of the mesoporous silica during C₃S synthesis, the formation of by-products such as calcium oxide and dicalcium silicate can be effectively suppressed. The dicalcium silicate content in the pulp capping material of the present invention is below 5 wt %, which significantly enhances the initial setting rate of the material and facilitates applications requiring rapid shaping and early demolding.

In another aspect, the pulp capping material of the present invention, in addition to containing the aforementioned tricalcium silicate, further comprises bioactive glass fibers and zirconium dioxide, which provide sufficient mechanical strength, and the initial setting time can be reduced to 7 minutes. In terms of biocompatibility testing, the material showed not only no significant cytotoxicity but also a remarkable improvement in long-term cell viability. To further investigate the inducing ability of cell remineralization in tricalcium silicate containing bioactive fibers, by alkaline phosphatase quantification analysis and gene expression analysis of DSPP and other related genes, also indicate that the pulp capping material of the present invention significantly enhances the production of alkaline phosphatase and the expression of DSPP in more differentiated dental pulp stem cells.

In accordance with one embodiment, the present invention provides a pulp capping material comprising tricalcium silicate, bioactive glass fiber, and a radiopacity material, wherein a precursor of the tricalcium silicate is mesoporous silica having an average pore diameter in the range of 10 nm to 50 nm.

In accordance with another embodiment, the present invention provides the pulp capping material as described above, wherein the average pore diameter of the mesoporous silica is in the range of 15 nm to 30 nm.

In accordance with another embodiment, the present invention provides the pulp capping material as described above, wherein the tricalcium silicate is present in an amount of 70 wt % to 85 wt % of the pulp capping material, the bioactive glass fiber is present in an amount of 5 wt % to 25 wt % of the pulp capping material, and the radiopacity material is present in an amount not exceeding 20 wt % of the pulp capping material.

In accordance with another embodiment, the present invention provides the pulp capping material as described above, wherein the tricalcium silicate is present in an amount of 75 wt % to 85 wt % of the pulp capping material, the bioactive glass fiber is present in an amount of 15 wt % to 25 wt % of the pulp capping material, and the radiopacity material is present in an amount not exceeding 15 wt % of the pulp capping material.

In accordance with another embodiment, the present invention provides the pulp capping material as described above, wherein when the pulp capping material is mixed with water at half the weight of the pulp capping material, an initial setting time does not exceed 10 minutes and a final setting time does not exceed 15 minutes.

In accordance with another embodiment, the present invention provides the pulp capping material as described above, wherein when the pulp capping material is mixed with water at half the weight of the pulp capping material, a diametral tensile strength after 3 days is between 5 and 7 MPa, and after 10 days is between 8 and 11 MPa.

In accordance with another embodiment, the present invention provides the pulp capping material as described above, wherein the amount of dicalcium silicate in the pulp capping material is less than 5 wt %.

In accordance with another embodiment, the present invention provides the pulp capping material as described above, wherein the pulp capping material is free of tricalcium aluminate.

In accordance with another embodiment, the present invention provides the pulp capping material as described above, wherein the pulp capping material is in powder form.

In accordance with another embodiment, the present invention provides the pulp capping material as described above, wherein the pulp capping material is a single-component formulation that only requires the addition of water for use in the oral cavity. Other embodiments of the present invention may also include pulp capping materials comprising excipients, catalysts, or adjuvants.

In accordance with another embodiment, the present invention provides the pulp capping material as described above, wherein the mesoporous silica is prepared by a sol-gel process and calcium is introduced via an impregnation process to form the tricalcium silicate.

In accordance with another embodiment, the present invention provides the pulp capping material as described above, wherein the sol-gel process includes the use of a metal-free silicon source to produce the mesoporous silica.

In accordance with another embodiment, the present invention provides the pulp capping material as described above, wherein when the tricalcium silicate is mixed with water at half the weight of the tricalcium silicate, an initial setting time is between 17 and 23 minutes and a final setting time is between 35 and 40 minutes.

In accordance with another embodiment, the present invention provides the pulp capping material as described above, wherein when the tricalcium silicate is mixed with water at half the weight of the tricalcium silicate, a diametral tensile strength after 3 days is between 4 and 6 MPa, and after 10 days is between 4.5 and 7 MPa.

In accordance with another embodiment, the present invention provides the pulp capping material as described above, wherein the calcium oxide content in the tricalcium silicate is less than 1%.

In accordance with another embodiment, the present invention provides the pulp capping material as described above, wherein the pulp capping material contains no additionally added calcium oxide.

In accordance with another embodiment, the present invention provides the pulp capping material as described above wherein the bioactive glass fiber comprises at least: silicon dioxide, sodium oxide or calcium oxide, and phosphorus pentoxide.

In accordance with another embodiment, the present invention provides the pulp capping material as described above, wherein the bioactive glass fiber comprises 60 wt % to 75 wt % of silicon dioxide, 30 wt % to 35 wt % of sodium oxide or calcium oxide, and no more than 10 wt % of phosphorus pentoxide.

In accordance with another embodiment, the present invention provides the pulp capping material as described above, wherein the diameter of the bioactive glass fiber is approximately 8 to 10 μm.

In accordance with another embodiment, the present invention provides the pulp capping material as described above, wherein the radiopacity material is zirconium dioxide.

The present invention also includes other aspects and various embodiments to solve other problems. These and other aspects are detailed in the implementation examples.

### BRIEF DESCRIPTIONS OF THE DRAWINGS

The invention will be further explained in more detail based on exemplary specific embodiments shown in the accompanying drawings, in which:

FIG. 1 is the X-ray diffraction (XRD) pattern of tricalcium silicate and the corresponding tricalcium silicate standard data file.

FIG. 2(A) and FIG. 2(B) respectively are the microstructure of tricalcium silicate as observed under electron microscopy; FIG. 2(A) is a scanning electron microscope (SEM) image, and FIG. 2(B) is a transmission electron microscope (TEM) image.

FIG. 3 is the energy-dispersive X-ray spectroscopy (EDS) analysis of tricalcium silicate.

FIG. 4 shows the diametral tensile strength test results of various pulp capping materials after 3 days and 10 days hydration (n=6) (different letters indicate statistically significant differences, p<0.05).

FIG. 5(A) and FIG. 5(B) are the cross-sectional microstructure of the M70-Z20-G10 specimen after hydration as observed under scanning electron microscopy. FIG. 5(A) is the specimen after 3 days of hydration, and FIG. 5(B) is after 10 days of hydration.

FIG. 6 shows the MTT assay results for human dental pulp stem cells cultured with extract solutions for 8 hours, 24 hours, and 72 hours (mean±standard error), (n=3) (different letters indicate statistically significant differences, p<0.05).

FIG. 7(A) and FIG. 7(B) show the activity of alkaline phosphatase in human dental pulp stem cells cultured with extract solutions for 7 days and 14 days, respectively (mean±standard error) (n=3) (different letters indicate statistically significant differences, p<0.05).

FIG. 8(A) and FIG. 8(B) are the gene expression levels of DSPP in human dental pulp stem cells cultured with extract solutions for 7 days and 14 days, respectively (mean±standard error) (n=3) (different letters indicate statistically significant differences, p<0.05).

### DETAILED DESCRIPTION OF THE INVENTION

The following description illustrates the method and apparatus for manufacturing the pulp capping material of the present invention through specific embodiments. Those skilled in the art can understand the advantages and effects of the present invention based on the disclosures provided herein. It should be understood that these different specific embodiments or examples are provided for illustrative purposes only and are not intended to limit the present invention. The invention may be implemented or applied through other various embodiments. Those skilled in the art may modify and/or alter the specific embodiments according to different implementations and applications without departing from the spirit and scope of the present invention.

### Tricalcium Silicate

The tricalcium silicate of the present invention is synthesized using a sol-gel process and an impregnation process. The sol-gel process is used to obtain mesoporous silica (also referred to as vesicle-like mesoporous silica) with a average pore diameter ranging from 15 nm to 30 nm, of which it involves the addition of a suitable template (such as the surfactant P123) to control the pore structure. The method can comprise first dispersing the template in water, followed by the addition of a silicon-containing organic compound (e.g., TEOS or other metal-free organosilicon compounds) to undergo hydrolysis and condensation reactions, forming a colloidal solution containing dispersed nanoparticles. As the reaction proceeds, the particles gradually crosslink into a three-dimensional network, and the liquid transforms into a solid gel. Subsequent drying is performed to remove the solvent, followed by high-temperature calcination to remove the template or other organic components and to reinforce the material structure, ultimately forming vesicle-like mesoporous silica. Next, using this vesicle-like mesoporous silica as a precursor, tricalcium silicate is further synthesized via the impregnation process. The method may comprise bringing a solution containing a calcium source or other functional components into full contact with the vesicle-like mesoporous silica, allowing it to penetrate into the pores, following by drying and thermal treatment to deposit the active components and fix them onto the surface or inside the pores of the mesoporous silica. In the present invention, a non-toxic mesoporous silica material with a porous surface is used as the precursor. The porous structure facilitates the binding of calcium with silicon, and the process is simpler and more conducive to obtaining purified tricalcium silicate. One disadvantage of tricalcium silicate is that its setting time is similar to the conventional pulp capping material, and its compressive strength is relatively low; therefore, additional components are required to enhance its mechanical strength.

### Bioactive Glass Fibers

The bioactive glass fibers used in the present invention are prepared by subjecting bioactive glass powder to high-temperature melting followed by fiber-forming treatment, then winding for fibrillation, and subsequently pulverizing to obtain a powder product. The bioactive glass powder is composed of specific ratios of silicon dioxide (SiO₂), sodium oxide (Na₂O), and/or calcium oxide (CaO), and phosphorus pentoxide (P₂O₅). It can bond with bone tissue due to the ability of its surface to accumulate a biologically active hydroxyapatite layer, and it does not induce the formation of excess fibrous tissue in the surrounding area. When applied in vital pulp therapy, bioactive glass produces cellular responses in dental pulp stem cells similar to those induced by the conventional pulp capping material, and can likewise promote the formation of reparative dentin. Moreover, in terms of shortened setting time and improved manual handling, bioactive glass-based materials offer advantages over the conventional pulp capping material.

### Radiopacity Material

According to one embodiment of the present invention, zirconium dioxide is used as the radiopacity material. Zirconium dioxide possesses good biocompatibility, osseointegration, and bioinertness. In vital pulp therapy materials, it not only provides sufficient radiopacity but also enhances strength, and thus can replace bismuth oxide, which tends to reduce strength by interfering with the hydration reaction. Compared to the conventional pulp capping material, Portland cement containing a certain proportion of zirconium dioxide exhibits superior physical properties such as hardness and compressive strength, and also results in a shorter setting time.

### Preparation and Characterization of Experimental Materials

### Mesoporous Tricalcium Silicate, MC3S

1.50 g of the triblock copolymer surfactant P123 was used as a soft template and dispersed in 600.00 g of double-distilled water, stirred for half a day until fully dissolved. Then, 12.00 g of TEOS was added and stirred in a closed reaction in a thermostatic water bath at 55° C. for 3 days. The reaction solution was transferred into a hydrothermal vessel and placed in an oven at 100° C. for hydrothermal reaction for 24 hours. The resulting product was filtered, dried in an oven at 70° C., and then placed in a high-temperature furnace for calcination. The temperature was ramped to 600° C. over 3 hours and held for 6 hours in air to remove the polymer surfactant and enhance structural integrity, forming vesicle-like mesoporous silica.

10.805 g of Ca(NO₃)₂ was dissolved in 100 g of double-distilled water to prepare a Ca(NO₃)_{2(aq)} solution. Then, 1.1 g of the synthesized vesicle-like mesoporous silica was added to the solution to form a mixture. The mixture was heated and stirred until it became a paste and could no longer be stirred. It was then calcined at 600° C. and 900° C. for 6 and 5 hours, respectively, to form a white powder. This powder was subsequently calcined at 1400° C. for 2 hours and immediately cooled. Finally, the powder was ground using a ball mill to a particle size of approximately <5 μm.

### X-Ray Diffraction (XRD) Analysis of Tricalcium Silicate

The synthesized powder was placed on the sample stage of an X-ray diffractometer (Rigaku, Ultima IV, USA) for analysis. The diffraction angles and intensities were compared with those in the standard database (International Centre for Diffraction Data) to confirm that the product matched the expected result.

### Transmission Electron Microscopy (TEM) Observation of Tricalcium Silicate

Scanning electron microscopy observation: The powder synthesized in section 3.1.1 was fixed onto a sample stage using conductive carbon tape, and a gold layer was coated under vacuum using a metal ion coater (Q150R coating machine, Quorum Technologies Ltd, United Kingdom) under the condition of 25 mA for 120 seconds. Then, the particle size and morphology of the sample were observed using a transmission electron microscope (Hitachi SU8220 STEM-TE detecting device).

### Synthesis of Bioactive Glass Fiber

The general method for synthesizing bioactive glass fibers involves feeding bioactive glass powder (composed of 65% SiO₂, 31% CaO, and 4% P₂O₅) into a high-temperature (approximately 1400° C.) natural gas furnace using a pneumatic conveyor for direct melting to remove excess bubbles and improve fluidity. Then, the molten glass was then directed into a fining machine and cooled to 1370° C. It was extruded through orifices in the forehearth sleeve into fiber form. Then, a high-speed winding machine was used to draw the molten glass under spinning tension into fibers. Lastly, the fibers were ground using a ball mill for 1.5 minutes. In this embodiment of the present invention, 63S bioactive glass fibers (produced by Great Sky Fiber Co., Ltd.) with a diameter of approximately 8-10 μm were used.

### Preparation of Bioactive Pulp Capping Material

The 63S bioactive glass fibers and 5 μm zirconium dioxide powder were added in different weight percentages to the aforementioned synthesized mesoporous tricalcium silicate (M-C3S) to form pulp capping materials (composition shown in the table below). The pulp capping materials were uniformly mixed with water at a ratio of M-C3S:ddwater=2:1. CP-1 and 2 (CP-1, CP-2) were used for comparison, as summarized below.

### Compositional Ratios of Experimental Samples

| | M—C3S | ZrO2 | 63S Fiber | Water/M—C3S |
|---|---|---|---|---|
| Sample | (wt. %) | (wt. %) | (wt. %) | Ratio |
| M100 | 100 | 0 | 0 | 1:2 |
| M80-Z20 | 80 | 20 | 0 | |
| M70-Z20-G10 | 70 | 20 | 10 | |

| | |
|---|---|
| CP-1 | C3S, C2S, C3A, CaO, ZrO2, SiO2, |
| | Polyethylene Glycol, Fe2O3 |
| CP-2 | C3S, CaCO3, ZrO2, CaCl2(aq) |

### Mechanical Property Analysis of the Materials

### Setting Time

According to ISO 9917 regulation, stainless steel molds with an inner diameter of 6 mm and a height of 2 mm were used. Each test material was mixed and filled into the molds, which were then placed in an environment with a temperature of 37° C. and 100% relative humidity. Subsequently, Gillmore needles were used to measure setting times. The initial setting needle and the final setting needle were gently applied to the surface of the material at 3-minute intervals until no indentation was observed. The corresponding time was recorded as the initial setting time or the final setting time, respectively. This test was repeated four times for each group.

### Diametral Tensile Strength (DTS) Test

Each group of materials was filled into stainless steel molds with an inner diameter of 6 mm and a height of 2 mm, and placed in an environment at 37° C. and 100% relative humidity. After hydration and setting for 3 days and 10 days, respectively, the cylindrical disc-shaped specimens were demolded and collected, and then subjected to diametral tensile strength (DTS) testing using a universal testing machine (Instron 5566, Instron, USA) (compressive load: 5 kN; crosshead speed: 0.5 mm/min; sample size: 6). The maximum load was recorded using Bluehill software, and the DTS was calculated using the following formula:

$$DTS = \frac{2 \times Maximum Load ( N )}{π \times Specimen Diameter ( mm ) \times Specimen Height}$$

### Observation of the Microstructure of the Materials by Scanning Electron Microscopy (SEM)

Following the diametral tensile strength (DTS) test, the fractured cross-sections of the specimens were fixed onto the sample holder using conductive carbon tape. A layer of gold (Au) ions was coated under vacuum using a metal ion coating machine (Q150R coating machine, Quorum Technologies Ltd, United Kingdom) under the conditions of 25 mA for 90 seconds. Subsequent cross-sectional observations were performed using a scanning electron microscope (Hitachi S-2400; Nova NanoSEM™ 30 Series, FEI, HK).

### In Vitro Cell Viability and Biocompatibility Testing

### Preparation of Extracts from Pulp Capping Materials

In accordance with ISO 10993-5, regarding the extract preparation protocol for testing the biocompatibility of dental materials (3.1.5), after each material was allowed to set for 10 days, the samples were immersed at a ratio of 33 mg/ml in α-MEM cell culture medium supplemented with 1% fetal bovine serum (FBS) and 1% penicillin/streptomycin, following by cultivating in an incubator at 37° C. with 5% CO₂ for 24 hours. After this extraction process was repeated three times, equal volumes of the resulting extracts were then combined and mixed with fresh α-MEM medium containing 1% FBS and 1% penicillin/streptomycin. Lastly, the extract solution was filtered through a 0.22 μm membrane filter and stored for subsequent use. Additionally, the control group used α-MEM culture medium supplemented with 1% fetal bovine serum (FBS) and 1% penicillin/streptomycin.

### Primary Culture of Human Dental Pulp Stem Cells

With informed consent obtained from the patients, non-carious and unrestored premolars and third molars were collected from the Department of Oral and Maxillofacial Surgery at National Taiwan University Hospital as sources of dental pulp tissue (IRB approval number: NTUH-58755). Immediately after tooth extraction, the samples were preserved in dental pulp stem cell (DPSC) culture medium. The teeth were then vertically split using a sterile chisel to expose and extract the intact pulp tissue. Tissue from the central portion of the pulp was cut into small fragments measuring approximately 1 mm×1 mm×1 mm, which were placed into 10 mL of culture medium and transferred to a 10 cm² culture dish for incubation. After approximately 7 to 10 days, dental pulp stem cells were observed to grow from the tissue fragments and adhere to the surface of the culture dish. When the cells reached appropriate confluence, they were passaged at a ratio of 1:3. Cells at passage 8 were used for subsequent experiments. The DPSC culture medium consisted of α-MEM supplemented with 10% fetal bovine serum (FBS) and 1% penicillin/streptomycin.

### MTT Cell Viability Assay

To evaluate the effect of the synthesized pulp capping materials on cell growth, the MTT (3-(4,5-dimethylthiazol-2-yl)-2,5-diphenyltetrazolium bromide) assay was used to measure cell proliferation and viability. This assay enables assessment of the effects of the test materials on cell growth. MTT is a yellow compound, used in the mitochondrial respiratory chain of viable cells, that can convert into purple formazan crystals under the action of succinate dehydrogenase (SDH) and cytochrome C. Using dimethyl sulfoxide (DMSO, Fisher Scientific, Hampton, NH, USA) to solubilize formazan, the amount of crystalized formazan produced is proportional to the number of viable cells. Cell viability and proliferation were evaluated by measuring absorbance using an ELISA reader, with absorbance corresponding to mitochondrial activity and cell number.

Human dental pulp stem cells were used as the experimental model and seeded into 96-well plates at a density of 5×10³ cells per well. After 24 hours of incubation, the original culture medium (α-MEM with 10% FBS) was removed and replaced with 100 μL/well of material extract. All cells were incubated at 37° C. with 5% CO₂ for 8 hours, 1 day, and 3 days, of which the 8-hour and 1-day timepoints were used to evaluate acute cytotoxicity (immediate cell viability), while the 3-days incubation assessed sustained cytotoxicity (long-term cell viability).

After removing the material extract, cells were washed with PBS, and 20 μL of MTT solution (0.5 mg/ml) was added to each well. The reaction was carried out at 37° C. in a 5% CO₂ incubator for 4 hours under dark. After incubation, the MTT solution was removed and 200 μL of DMSO was added to each well. Absorbance was measured at 570 nm using an ELISA reader. All optical density (OD) values were background-corrected and the data were collected in triplicate. The cell viability was calculated using the following formula:

$$\% Cell Viability = \frac{OD ( experimental group ) - OD ( blank )}{OD ( control group ) - OD ( blank )} \times 100 \%$$

### Analysis of In Vitro Cellular Mineralization Behavior

### ALP Activity Quantitative Assay

This quantitative assay is based on the principle that alkaline phosphatase (ALP) catalyzes the hydrolysis of p-nitrophenyl phosphate (p-NPP) to release phosphate and the yellow p-nitrophenol, of which the amount of p-nitrophenol produced is directly proportional to the ALP activity present in the reaction. Therefore, quantification was performed by measuring the absorbance at 405 nm using a spectrophotometer. In this study, the Quantitative Alkaline Phosphatase ES Characterization Kit (Millipore) was used.

Human dental pulp stem cells (1×10⁵) were seeded in two 6-well culture plates. After 24 hours of incubation until the cell adhesion, 2 mL of material extract or control culture medium was added to each well and incubated at 37° C. The extract and control medium were refreshed every three days. ALP activity was analyzed on days 7 and 14.

After removing the cultural medium, the cells were washed with phosphate-buffered saline (PBS), followed by adding with the dissociation enzyme Accutase (Cat. No. SCR005) to detach the cells. The detached cells were collected in 15 ml conical centrifuge tubes and centrifuged at 2000 rpm for 5 minutes. After centrifugation, the supernatant was discarded, and the cell pellet was resuspended in 2 mL of 1× Wash Solution. The cells were then counted. From both the experimental and control groups, 60,000 cells were collected and evenly distributed into three Eppendorf tubes for repeating sample testing. The tubes were centrifuged again at 2000 rpm for 5 minutes at room temperature and reserved for subsequent analysis.

Each Eppendorf tube was resuspended in 50 μL of p-NPP buffer, and the contents were transferred into individual wells of a 96-well plate. The enzymatic reaction was initiated by adding 50 μL of 2× p-NPP Substrate Solution to each well, followed by incubation in the dark at room temperature for 20 minutes. The reaction was terminated by adding 50 μL of Reaction Stop Solution to each well, and the absorbance (optical density, OD) was measured at 405 nm using an ELISA reader. In addition, a standard curve was generated using a set of p-NPP standards at the following concentrations: 10, 5, 2.5, 1.25, 0.625, 0.3125, 0.15625, and 0 ng/ml.

### Analysis of Mineralization-Related Gene Expression: Quantitative Real-Time PCR

Total RNA was reverse transcribed into complementary DNA (cDNA) by reverse transcriptase, serving as the template for subsequent qRT-PCR analysis of gene expression levels.

### RNA Extraction

Human dental pulp stem cells were seeded in 10 cm Petri dishes. After 24-hour cultivation, material extracts were added. The extract was refreshed every three days. On days 7 and 14, approximately 1×10⁶ cells were collected from each sample for RNA extraction using the RNeasy® Mini Kit.

For each sample, 350 μL of Buffer RLT was added, followed by an equal volume of 70% ethanol. A total of 700 μL of the mixture was then transferred to a RNeasy Mini spin column placed in a 2 mL collection tube and centrifuged at 8000 g for 15 seconds. The flow-through was discarded.

Next, 700 μL of Buffer RW1 was added to the spin column and centrifuged at 8000 g for 15 seconds. After discarding the flow-through, 500 μL of Buffer RPE was added, centrifuged under the same conditions for 15 min and removed the flow-through. This RPE wash step was repeated once more.

Next, the RNeasy spin column was transferred to a new 1.5 mL collection tube, and 30-50 μL of RNase-free water was added. The column was centrifuged at 8000 g for 60 seconds to elute purified RNA.

Lastly, the RNA concentration and purity were assessed using an ELISA reader to measure absorbance at 260/280 nm, ensuring values between 1.8 and 2.0. The final RNA concentration was adjusted to 100 μg/mL, confirming successful extraction of high-quality RNA.

### cDNA Synthesis

cDNA was synthesized using the iScript cDNA Synthesis Kit according to the manufacturer's protocol to prepare 20 μL reaction for each sample group containing: 4 μL of 5× iScript reaction mix; 1 μL of iScript reverse transcriptase; 5 μL of RNase-free water; 10 μL of RNA sample.

Thermal cycling was carried out as follows: 25° C. for 5 minutes (priming), 46° C. for 20 minutes (reverse transcription), and 95° C. for 1 minute (reaction inactivation). These steps ensured efficient reverse transcription of RNA into cDNA for subsequent analysis and application.

### qRT-PCR

Prior to the qRT-PCR experiment, the following preparation steps were carried out in advance. First, DNA samples (see group 3.3.1) were adjusted to 100 ng/μL. Next, forward and reverse primers were prepared at 10 μM concentrations. The target gene was DENTIN SIALOPHOSPHOPROTEIN (DSPP), with GAPDH used as the internal reference gene. The primer sequences are listed in Table 3-2.

TABLE 3-2
lists the forward and reverse primers used for the mineralization-related gene
and the internal control gene. The contents of the electronic sequence listing
(US20260007580_Sequence-listings-for-filing_VP135656-010100; Size: 5k bytes; and
Date of Creation: Jul. 18, 2025) are herein incorporated by reference in its
entirety.

| | Forward Primer Sequence (5′-3′) | Reverse Primer Sequence (5′-3′) |
|---|---|---|
| DSPP | TGCATTTGGGCAGTAGCATGG | TTCATGCACCAGGACACCATT |
| | (Sequence ID No.: 1) | (Sequence ID No.: 2) |
| GAPDH | AGGGACCTGGTATGTTCTCCT | CCAGCTTCCTGTAGCACTCAA |
| | (Sequence ID No.: 3) | (Sequence ID No.: 4) |

On ice, a 10 μL reaction mixture was prepared for each well of a 96-well plate: 0.25 μL of forward and reverse primer, 1 μL of DNA template, 5 μL of iTaq Universal SYBR Green Supermix, and 3.5 μL of Nuclease-free water. The plate was sealed with a specialized optical adhesive film to prevent contamination and sample evaporation.

qRT-PCR cycling conditions were set as follows: 95° C., 10 seconds; 60° C., 30 seconds, repeated for 40 cycles; final extension: 95° C., 15 seconds, 60° C., 1 min, and 95° C., 15 sec.

The relative expression levels of mineralization gene were calculated using the 2^{-ΔΔCt} method. This approach enables relative quantification of target gene expression differences between sample.

### Statistical Analysis

Statistical comparisons for DTS, MTT assay, ALP quantification, and qRT-PCR results were conducted using SPSS 25.0 software. One-way ANOVA followed by the Least Significant Difference (LSD) test was applied. A p-value <0.05 was considered statistically significant.

### Experimental Results

### X-Ray Diffraction (XRD) Analysis of Tricalcium Silicate

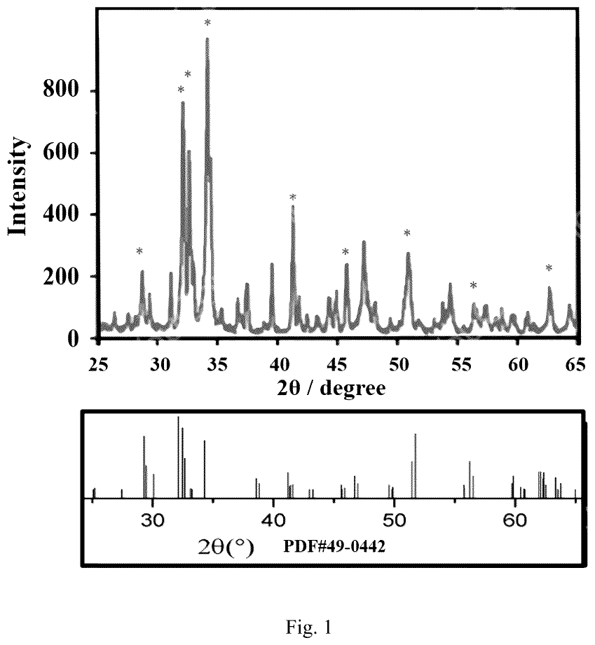

FIG. 1 shows the XRD pattern of tricalcium silicate. Comparison with the reference diffraction data file PDF #49-0442 from the International Center for Diffraction Data (ICDD) confirmed that the major characteristic peaks matched the standard pattern.

### Microstructure of Tricalcium Silicate

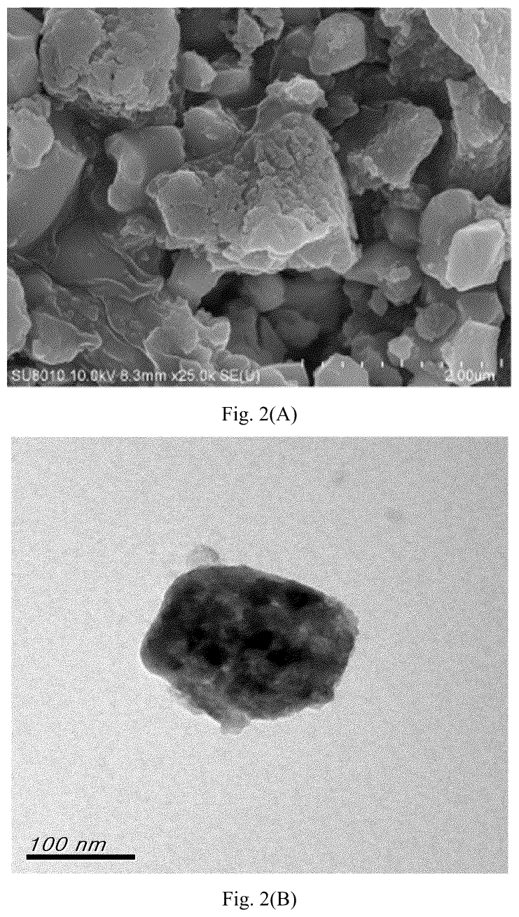

FIG. 2 presents the microstructure of the synthesized tricalcium silicate (MC3S), as observed under scanning electron microscopy (SEM) and transmission electron microscopy (TEM).

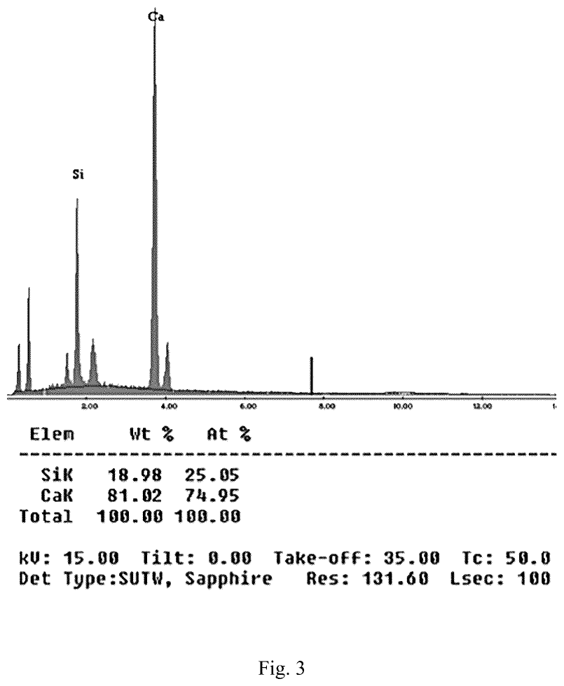

FIG. 3 shows the elemental composition of tricalcium silicate as analyzed by EDS, revealing a calcium-to-silicon ratio of 3:1, confirming the successful synthesis of mesoporous tricalcium silicate.

### Setting Time Test

The initial setting time of MC3S containing bioactive glass fiber and zirconium dioxide (M70-Z20-G10) was 7 minutes, and the final setting time was 13.5 minutes, which was far shorter than the commercial controls (CP-1, CP-2), as shown in the table below.

### Results of Setting Time Tests for the Materials (n=3)

| | Initial Setting Time | Final Setting Time |
|---|---|---|
| | (min, mean ± | (min, mean ± |
| Materials | standard error) | standard error) |
| CP-1 | 89.0 ± 6.0 | 180.0 ± 9.5 |
| CP-2 | 13 ± 0.7 | 20 ± 0.7 |
| M100 | 20 ± 2.5 | 38 ± 1.0 |
| M80-Z20 | 11.5 ± 1.5 | 35 ± 1.5 |
| M70-Z20-G10 | 7.0 ± 1.2 | 13.5 ± 0.6 |

### Diametral Tensile Strength (DTS) Test

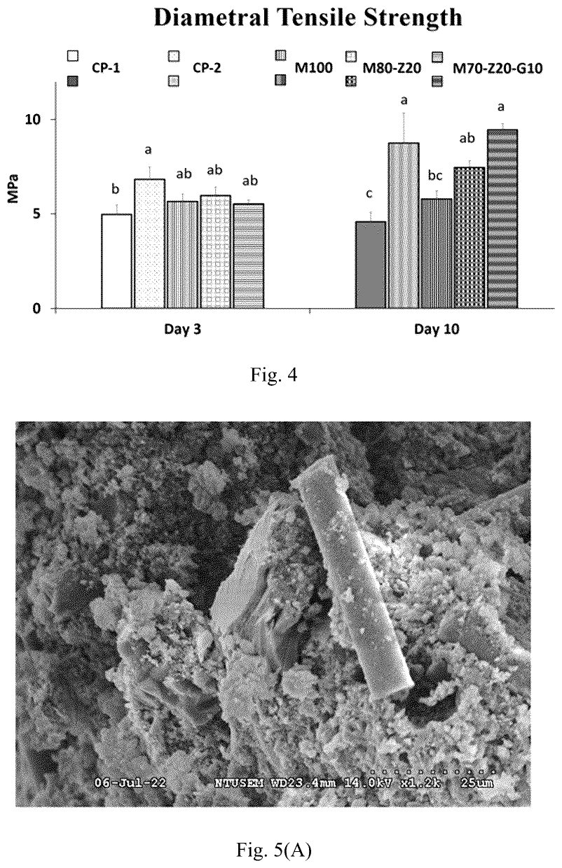

FIG. 4 shows the DTS values of each pulp capping material after 3 and 10 days of hydration. All groups exhibited increased strength at 10 days compared to 3 days, except for CP-1, whose strength did not increase with hydration time (3 days: 4.95±0.53 MPa; 10 days: 4.57±0.51 MPa).

On 3 days of hydration, CP-2 (6.81±0.67 MPa) exhibited significantly higher strength than CP-1 (p<0.05), while no significant differences were observed among the other groups. On 10 days, M70-Z20-G10 showed the highest strength at 9.44±0.34 MPa, 1.7 times its strength at day 3 (5.51±0.23 MPa). Compared with commercial products, M70-Z20-G10 and M80-Z20 (7.46±0.37 MPa) showed no significant difference from CP-2 (8.74±1.62 MPa), but were significantly stronger than CP-1 (p<0.05).

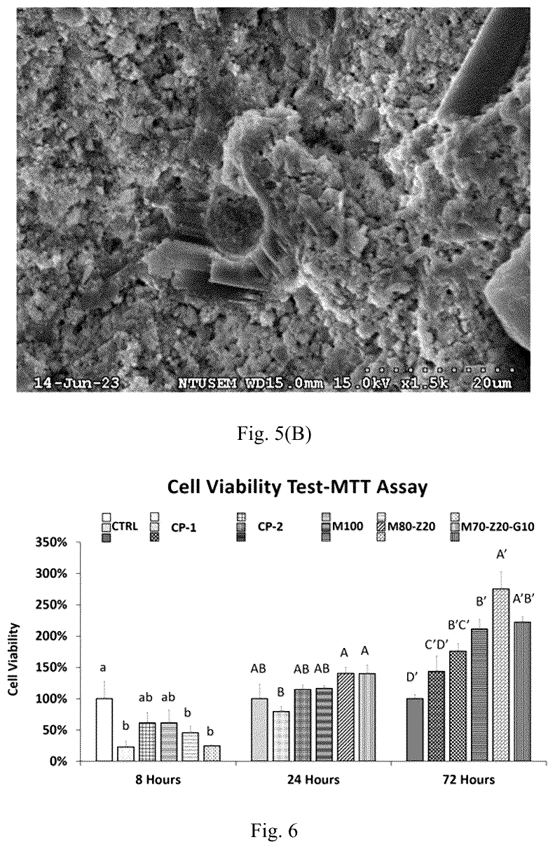

FIG. 5(A) and FIG. 5(B) show the cross-sectional microstructures of M70-Z20-G10 specimens after 3 and 10 days of hydration, respectively, as observed by SEM. The bioactive glass fibers served as reinforcement scaffolds for M-C3S and exhibited co-crystallization at both timepoints.

### In Vitro Biocompatibility Test (MTT Cell Viability Assay)

FIG. 6 presents the MTT assay results of dental pulp stem cells cultured with different extract media for 8, 24, and 72 hours. The groups included α-MEM control, CP-1 extract, CP-2 extract, M100 extract, M80-Z20 extract, and M70-Z20-G10 extract. At 8 hours, no significant differences in CP-1 extract, CP-2 extract, M100 extract, M80-Z20 extract, and M70-Z20-G10 extract were observed among the five experimental groups, while the α-MEM control group showed the highest cell viability.

At 24 hours, the testing results of MTT testing can be observed: M80-Z20 and M70-Z20-G10 extracts showed the highest cell viability, with significantly better results compared to CP-1 (p<0.05).

At 72 hours, M80-Z20 extract yielded the highest cell viability, significantly outperforming the α-MEM control, CP-1, and CP-2 (p<0.05), though there was no significant difference compared to M70-Z20-G10 which included bioactive glass fibers.

Additionally, based on average values, the M100, M80-Z20, and M70-Z20-G10 extracts ranked as the top three in cell viability at both 24 and 72 hours.

### Analysis of In Vitro Mineralization Behavior: Quantification of Alkaline Phosphatase (ALP)

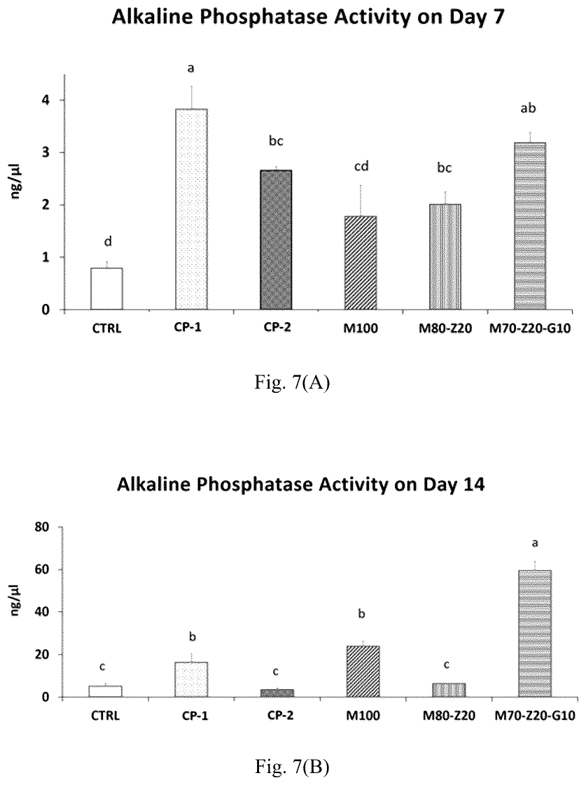

FIG. 7(A) and FIG. 7(B) show the ALP quantification results of dental pulp stem cells cultured with different extracts for 7 and 14 days, respectively. At day 14, the M70-Z20-G10 group, which contains bioactive glass fiber, exhibited the highest ALP activity. While ALP levels increased over time in all groups, the increase in M70-Z20-G10 was the most pronounced.

On day 7, among sample groups of cell culture with extracts, CP-1 (3.83±0.44 ng/μL) and M70-Z20-G10 (3.18±0.2 ng/μL) showed the highest ALP levels, both significantly higher than the control (p<0.05), with no significant difference between them. On day 14, M70-Z20-G10 (59.51±4.49 ng/μL) significantly outperformed all commercial products and the control group (p<0.05).

### Gene Expression Analysis of Mineralization-Related Genes

### DSPP Gene

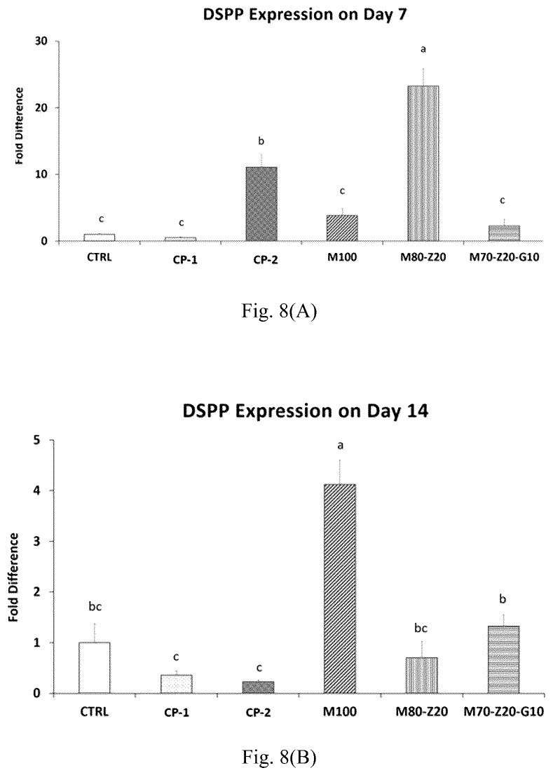

As shown in FIG. 8(A), DSPP expression in cells cultured for 7 days with M80-Z20 extract (23.24±2.65) was significantly higher than that of other groups. However, at 14 days, FIG. 8(B) shows that at the later stage of cellular differentiation, while other commercial groups exhibited downregulation, M100 (4.12±0.48) and M70-Z20-G10 (1.33±0.23) maintained positive regulation and were significantly higher than the commercial groups.

The above descriptions are preferred embodiments of the invention and are not intended to limit the scope of the patent application. Equivalent changes or modifications made without departing from the spirit of the invention should be included within the scope of the following patent claims.

What is claimed is:

1. A pulp capping material comprising tricalcium silicate, bioactive glass fiber, and a radiopacity material, wherein a precursor of the tricalcium silicate is mesoporous silica having an average pore diameter in the range of 10 nm to 50 nm.

2. The pulp capping material of claim 1, wherein the average pore diameter of the mesoporous silica is in the range of 15 nm to 30 nm.

3. The pulp capping material of claim 1, wherein the tricalcium silicate is present in an amount of 70 wt % to 85 wt % of the pulp capping material, the bioactive glass fiber is present in an amount of 5 wt % to 25 wt % of the pulp capping material, and the radiopacity material is present in an amount not exceeding 20 wt % of the pulp capping material.

4. The pulp capping material of claim 3, wherein the tricalcium silicate is present in an amount of 75 wt % to 85 wt % of the pulp capping material, the bioactive glass fiber is present in an amount of 15 wt % to 25 wt % of the pulp capping material, and the radiopacity material is present in an amount not exceeding 15 wt % of the pulp capping material.

5. The pulp capping material of claim 1, wherein when the pulp capping material is mixed with water at half the weight of the pulp capping material, an initial setting time does not exceed 10 minutes and a final setting time does not exceed 15 minutes.

6. The pulp capping material of claim 1, wherein when the pulp capping material is mixed with water at half the weight of the pulp capping material, a diametral tensile strength after 3 days is between 5 and 7 MPa, and after 10 days is between 8 and 11 MPa.

7. The pulp capping material of claim 1, wherein the amount of dicalcium silicate in the pulp capping material is less than 5 wt %.

8. The pulp capping material of claim 1, wherein the pulp capping material is free of tricalcium aluminate.

9. The pulp capping material of claim 1, wherein the pulp capping material is in powder form.

10. The pulp capping material of claim 1, wherein the pulp capping material is a single-component formulation that only requires the addition of water for use in the oral cavity.

11. The pulp capping material of claim 1, wherein the mesoporous silica is prepared by a sol-gel process and calcium is introduced via an impregnation process to form the tricalcium silicate.

12. The pulp capping material of claim 11, wherein the sol-gel process includes the use of a metal-free silicon source to produce the mesoporous silica.

13. The pulp capping material of claim 1, wherein when the tricalcium silicate is mixed with water at half the weight of the tricalcium silicate, an initial setting time is between 17 and 23 minutes and a final setting time is between 35 and 40 minutes.

14. The pulp capping material of claim 1, wherein when the tricalcium silicate is mixed with water at half the weight of the tricalcium silicate, a diametral tensile strength after 3 days is between 4 and 6 MPa, and after 10 days is between 4.5 and 7 MPa.

15. The pulp capping material of claim 1, wherein the calcium oxide content in the tricalcium silicate is less than 1%.

16. The pulp capping material of claim 1, wherein the pulp capping material contains no additionally added calcium oxide.

17. The pulp capping material of claim 1, wherein the bioactive glass fiber comprises at least silicon dioxide, sodium oxide or calcium oxide, and phosphorus pentoxide.

18. The pulp capping material of claim 1, wherein the bioactive glass fiber comprises 60 wt % to 75 wt % of silicon dioxide, 30 wt % to 35 wt % of sodium oxide or calcium oxide, and no more than 10 wt % of phosphorus pentoxide.

19. The pulp capping material of claim 1, wherein the diameter of the bioactive glass fiber is approximately 8 to 10 μm.

20. The pulp capping material of claim 1, wherein the radiopacity material is zirconium dioxide.


SEQUENCE LISTING (4 sequences)

SEQ ID NO: 1 - DNA, 21 bp, synthetic construct
  source 1..21: mol_type = other DNA
tgcatttgggcagtagcatgg

SEQ ID NO: 2 - DNA, 21 bp, synthetic construct
  source 1..21: mol_type = other DNA
ttcatgcaccaggacaccatt

SEQ ID NO: 3 - DNA, 21 bp, synthetic construct
  source 1..21: mol_type = other DNA
agggacctggtatgttctcct

SEQ ID NO: 4 - DNA, 21 bp, synthetic construct
  source 1..21: mol_type = other DNA
ccagcttcctgtagcactcaa